(19) United States
(12) Patent Application Publication
(10) Pub. No.: US 2010/0185082 A1
(43) Pub. Date: Jul. 22, 2010

(54) DEVICE AND METHOD FOR ELECTROSURGERY

(75) Inventors: Subashini CHANDRAN, Mississauga (CA); Krishnan SHAH, Mississauga (CA); Amy LEFLER, Orangeville (CA); Neil GODARA, Milton (CA); Robert HARRISON, Milton (CA)

Correspondence Address: Stephen E. Bondura;Dority & Manning, P.A., P.O. Box 1449, Greenville, SC, 29602, US

(73) Assignee: BAYLIS MEDICAL COMPANY INC., Montreal (CA)

(21) Appl. No.: 12/563,699

(22) Filed: Sep. 21, 2009

Related U.S. Application Data
(63) Continuation-in-part of application No. 11/079,318, filed on Mar. 15, 2005, now Pat. No. 7,593,778.
Application No. 11/079,318 is a continuation-in-part of application No. 10/382,836, filed on Mar. 7, 2003, now abandoned.

(51) Int. Cl. A61B 18/18; A61B 6/00

(52) U.S. Cl. 600/424; 606/33

(57) ABSTRACT

A method and apparatus are disclosed for improving accuracy of placement of a cannula during delivery of electrical energy to neural structures. The apparatus includes a cannula operable to deliver electrical current where a portion of the cannula is electrically insulated and a portion of the cannula is exposed and electrically conductive. The cannula further includes a radiopaque marker located to differentiate the electrically insulated region from the electrically exposed region by allowing it to be more clearly delineated using fluoroscopy or other radiographic imaging techniques. The cannula may be used to treat pain by delivering energy to a neural structure.

### CROSS-REFERENCE TO RELATED APPLICATIONS

This application is a continuation-in-part of U.S. patent application Ser. No. 11/079,318, filed on Mar. 15, 2005 which is a continuation-in-part of U.S. patent application Ser. No. 10/382,836, filed on Mar. 7, 2003.

### TECHNICAL FIELD

The invention relates to an electrosurgical device and more specifically to a device that can be used in the treatment of pain through the application of energy.

### BACKGROUND OF THE ART

Chronic back pain is a cause for concern throughout the world, including the United States, affecting as many as 80% of all Americans at some point in their lives. Lower back pain can arise from any number of sources, including but not limited to conditions of the spinal vertebrae themselves, the intervertebral disks and the facet joints of the spine. Although the precise cause of back pain is still a matter of debate, it is recognized that nerves present in these structures contribute to the sensation and transmission of these pain signals. Some of the recent advances in the treatment of back pain, therefore, have focused on treating the nerves deemed to be contributing to the pain sensations.

A minimally invasive technique of delivering high frequency electrical current has been shown to relieve localized pain in many patients. The high frequency electrical current is typically delivered from a generator via one or more electrodes that are placed in a patient's body. Resistance to the high frequency electrical current at the tip of the electrode causes heating of adjacent tissue and when the temperature increases sufficiently, the tissue coagulates. The temperature that is sufficient to coagulate unmyelinated nerve structures is 45° C., at which point a lesion is formed and pain signals are blocked. This procedure is known as tissue denervation and it usually results in significant pain relief. Radio frequency (RF) denervation refers to tissue denervation using energy in the RF range. This technique has proven especially beneficial in the treatment of back pain and more specifically, lower back pain. The field of medicine that relates to the lesioning of neural structures for the purpose of reducing pain is known as “pain management”. In this disclosure the term is used to describe the lesioning of neural structures to reduce pain.

U.S. Pat. No. 6,736,835 B2, issued May 18, 2004, U.S. Pat. No. 5,571,147, issued Nov. 5, 1996 and PCT patent application WO 01/45579 A1, published Jun. 28, 2001, all of which are incorporated herein by reference, amongst others, disclose methods and devices for performing RF denervation of various tissues of the back, including spinal vertebrae, intervertebral disks and facet joints of the spine. In general, the procedure entails introduction of an electrosurgical device into the body, positioning the device proximate to the neural tissue to be treated and applying RF electrical energy in order to denervate the tissue.

More specifically, an electrosurgical device comprising a cannula having a hollow shaft and a removable stylet therein is inserted into a patient's body and positioned at a desired treatment site. The cannula typically comprises an elongate, insulated region, along with an electrically conductive and exposed distal tip. Once the distal tip of the cannula is in position, the stylet is withdrawn and the distal end of a probe capable of delivering high frequency electrical energy is inserted until the distal end of the probe is at least flush with the exposed distal tip of the cannula. The proximal end of the probe is connected to a signal generator capable of generating high frequency electrical current. Once the distal end of the probe is in position, energy is supplied by the generator via the probe to denervate the tissue proximate to the distal end of the probe.

Accurate placement of the cannula requires significant technical skill and is a crucial aspect of any denervation procedure. If the cannula, and through it the probe, is positioned incorrectly, the results for the patient can be disastrous, as the denervating energy may be applied to a region of tissue that should not be denervated.

In order to facilitate accurate localization of the cannula in tissue denervation procedures, X-ray fluoroscopy is used to observe the cannula and to help guide the cannula through the body. Contrast in fluoroscopic images is achieved by means of variation in the absorbance of X-rays amongst different materials. Materials that are relatively radiopaque, such as bones and most metals, appear darker on fluoroscopic images, in contrast to the relatively radiolucent soft tissues of the body. One limitation of the technique used currently for RF denervation is that the insulated shaft of the cannula is indistinguishable from the exposed distal tip of the cannula under X-ray fluoroscopy, due to the fact that the entire cannula, i.e. both the insulated as well as the exposed regions, is generally made up of a radiopaque substance. Therefore, precise localization of the conductive distal tip of the cannula is very difficult as the entire cannula, comprising both the tip and the shaft, appears dark on the fluoroscopic image. In practice, doctors using tissue denervation to treat pain believe that the conventional method of positioning a conventional needle without additional radiopaque markers by conventional methods is sufficiently precise to generate the desired lesions in the target location and do not consider there to be any value in adding radiopaque markers to the electrical needles used in the lesioning of nerves. Conventional methods to position the active tip portion include visualizing a portion of the needle or visualizing the needle as a whole by interpreting the available fluoroscopic images. Improved specific localization of the distal tip of the cannula would be desirable as it is this region of the cannula that is electrically exposed and is therefore responsible for creating the lesion in the tissue.

In addition to fluoroscopy, two tests are typically conducted to confirm proximity to the target nerve and to confirm that the probe is not in proximity to other nerves prior to denervation. To assess proximity to the target nerve, an electrical stimulation is applied to the probe using a frequency that excites sensory nerves, typically 50 Hz with a current of up to 1 mA. A positive stimulation result reproduces the patient's pain, without producing other sensory responses in the lower extremity or buttocks. To confirm that the probe is not in proximity to an untargeted nerve, motor nerve stimulation is performed typically at a frequency of 2 Hz and a current of 3-5 mA. In this test, a lack of elicited muscle twitch in the lower limbs confirms that the probe is not at an undesired location near a spinal nerve. In the case of negative stimulation results, where there is a failure to reproduce the patient's pain or there is clear sensory or motor stimulation of the lower extremities, denervation is not performed. Rather, the probe is repositioned and proximity testing is repeated. Providing a manner of distinguishing the conductive distal tip of the cannula in fluoroscopic images may facilitate more accurate initial placement of the distal tip and avoid the requirement for probe repositioning.

Specifically with respect to facet joint denervation, positioning the cannula at the facet joint often requires the surgeon to steer or otherwise manipulate the trajectory of the device around a neural structure known as the sympathetic chain. The sympathetic chain refers to either of the pair of ganglionated longitudinal cords of the sympathetic nervous system of which one is situated on each side of the spinal column. Due to the proximity of the sympathetic chain which carries nerves that are critical to bodily function, facet joint denervation is a specific example of a procedure that may benefit from a manner of distinguishing portions of the cannula upon insertion in the body. The clinical success rate of this procedure ranges from 9% (Lora & Long, 1976) to 83% (Ogsbury et al., 1977). The wide range of success rates is thought to be chiefly due to variability in positioning the electrode and the resulting lesion relative to the target nerve, even when using fluoroscopy and stimulation pulses. An improvement in technique and apparatus for positioning the cannula, and through it the electrical probe, proximate to the facet nerve may increase the success rate of this procedure and reduce improper prone positioning as a reason for poor success.

The incorporation of radiopaque markers onto surgical devices has been used to increase the visibility of such devices under x-ray fluoroscopy. While techniques vary for producing and incorporating radiopaque markers onto surgical devices, the general concept involves incorporating a material with high x-ray absorption onto a specified medical device. U.S. Pat. No. 5,429,597, issued Jul. 4, 1995, which is incorporated herein by reference, discloses a balloon catheter having a radiopaque distal tip composed of a polymer mixed with a radiopaque powder sued as tungsten. U.S. Pat. No. 6,315,790 B1, issued Nov. 13, 2001, which is incorporated herein by reference, describes a stent delivery system having a catheter constructed with radiopaque polymer hubs wherein the hubs accomplish the dual functions of stent crimping and radiopaque marking. Another example of a catheter with a radiopaque marker is described in U.S. Pat. No. 5,759,174, issued Jun. 2, 1998, which is incorporated herein by reference. This catheter has a single external metal marker band used to identify the central portion of the stenosis once the delivery catheter is removed.

In all of the references noted above, radiopaque markers have been applied or attached to non-radiopaque devices, such as plastic or silicone-based catheters, and not to radiopaque devices such as metallic cannulae or needles.

### BRIEF DESCRIPTION OF THE DRAWINGS

In order that the invention may be readily understood, embodiments of the invention are illustrated by way of possible examples in the accompanying drawings, in which:

FIG. 1A is a top view of an embodiment of a radiopaque cannula with a radiopaque marker;

FIG. 1B is a sectional side view of the cannula depicted in FIG. 1A;

FIGS. 2 to 4 are top views illustrating alternate embodiments of a radiopaque cannula with associated radiopaque markers;

FIG. 5A is a bottom view of an embodiment of a radiopaque cannula comprising a rigidly bent distal portion;

FIG. 5B is a sectional side view of the cannula depicted in FIG. 6A;

FIG. 6 is a top view of an embodiment of a radiopaque cannula comprising a stylet;

FIG. 7 is a schematic illustration of an embodiment of a radiopaque cannula comprising a radiopaque marker connected to a high frequency generator;

FIG. 8 is a schematic diagram illustrating, in two different orientations, projected dimensions of an active tip and an associated radiopaque marker of a radiopaque cannula;

FIG. 9A is a lateral view showing a medial branch nerve and a cannula active tip positioned relative to the cervical vertebrae of a patient's spine;

FIG. 9B is an oblique anterior-posterior view showing a medial branch nerve and a cannula active tip relative to the lumbar vertebrae of a patient's spine; and

FIG. 9C is an anterior-posterior view showing a medial branch nerve and a cannula active tip relative to the thoracic vertebrae of a patient's spine.

### DETAILED DESCRIPTION

The disclosure is directed to a cannula for insertion into a patient's body and a method for treating pain using the cannula.

In accordance with a first aspect of the disclosure, a cannula for insertion into a patient's body is provided. The cannula comprises a radiopaque and electrically conductive elongate member having a proximal end, a distal end and a lumen defined therebetween. The elongate member includes an electrically exposed region and an electrically insulated region. A radiopaque marker is associated with the elongate member, the radiopaque marker being visible under radiographic imaging and located for distinguishing the exposed region from the insulated region.

As a feature of this aspect of the disclosure, the radiopaque marker is located substantially adjacent a distal end of the insulated region. In addition, the radiopaque marker comprises a material optionally selected from the group consisting of platinum, iridium, gold, silver, tantalum, palladium and alloys thereof. Additional possible features include that: at least a portion of the distal end of the elongate member is bent; the distal end of the elongate member defines an aperture in communication with the lumen; the elongate member defines a lateral aperture in communication with the lumen; the cannula is operable to connect to an energy source; and the electrically insulated region comprises a coating of an electrically insulating material disposed external to the elongate member along at least a portion of the elongate member.

Other additional possible features include that: the electrically insulating material comprises a material selected from the group consisting of parylene, PET and PTFE; the marker is affixed to the elongate member by a laser weld; the radiopaque marker comprises a band that substantially completely circumscribes the elongate member; the radiopaque marker comprises a material of a greater radiopacity than the material comprising the elongate member; and/or the elongate member is substantially rigid.

According to a second aspect of the disclosure, a cannula for insertion into a patient's body is provided. The cannula optionally comprises an electrically conductive radiopaque elongate member comprising an electrically insulated region and an electrically exposed region, and a means for improving radiographic visualization of at least a portion of the elongate member. The means for improving radiographic visualization is located for distinguishing the exposed region from the insulated region and can be external to and disposed along an intermediate position of the elongate member.

In accordance with a third aspect of the disclosure, a method for treating pain by delivering energy to a patient's body is described. The method comprises the following: (i) providing an electrically conductive radiopaque cannula comprising an electrically insulated region, an electrically exposed region and a radiopaque marker for distinguishing the insulated region from the exposed region; (ii) identifying the radiopaque marker, distinguishing the insulated region from the exposed region and positioning the exposed region to locate the exposed region proximate a neural structure; and (iii) delivering energy through the cannula to the neural structure to treat pain.

Additional possible features of the method include that: the neural structure is located proximate to or within a facet joint of a patient's spine; the energy is delivered directly to the neural structure to ablate the neural structure; a position of said electrically insulated region relative to said electrically exposed region is fixed; step (ii) further comprises positioning the exposed region substantially parallel and adjacent to one neural structure; step (ii) further comprises visualizing one or more anatomical landmarks associated with a location of the neural structure to facilitate positioning the exposed region to locate the exposed region proximate to the neural structure; and/or the method is used for allowing a user to determine the orientation of the cannula by visualizing the exposed region and the radiopaque marker.

The method can optionally further comprise: that in step (iii) the energy is delivered to create a first lesion and: (iv) determining the locations of a proximal end and of a distal end of the electrically exposed region; (v) either repositioning the cannula such that the distal end of the electrically exposed region is positioned substantially at the location in which the proximal end of the electrically exposed region had been determined to be located in step (iv) or repositioning the cannula such that the proximal end of the electrically exposed region is positioned substantially at the location in which the distal end of the electrically exposed region had been determined to be located in step (iv); and (vi) delivering energy through the cannula to the neural structure to create a second lesion.

Thus, the disclosure describes a novel cannula and methods of use thereof, wherein the cannula incorporates a means for improving radiographic visualization, for example a radiopaque marker, to allow for distinguishing an electrically exposed region of the cannula from an electrically insulated region utilizing radiographic imaging techniques such as fluoroscopy.

These features and others will become apparent in the detailed description that follows.

With specific reference now to the drawings in detail, it is stressed that the particulars shown are by way of example and for purposes of illustrative discussion of certain embodiments of the present invention only, and are presented in the cause of providing what is believed to be the most useful and readily understood description of the principles and conceptual aspects of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It should be noted that in this disclosure the term “pain management” is used to describe the field of medicine that relates to the lesioning of neural structures, located, for example, in the spine, for the purpose of reducing pain.

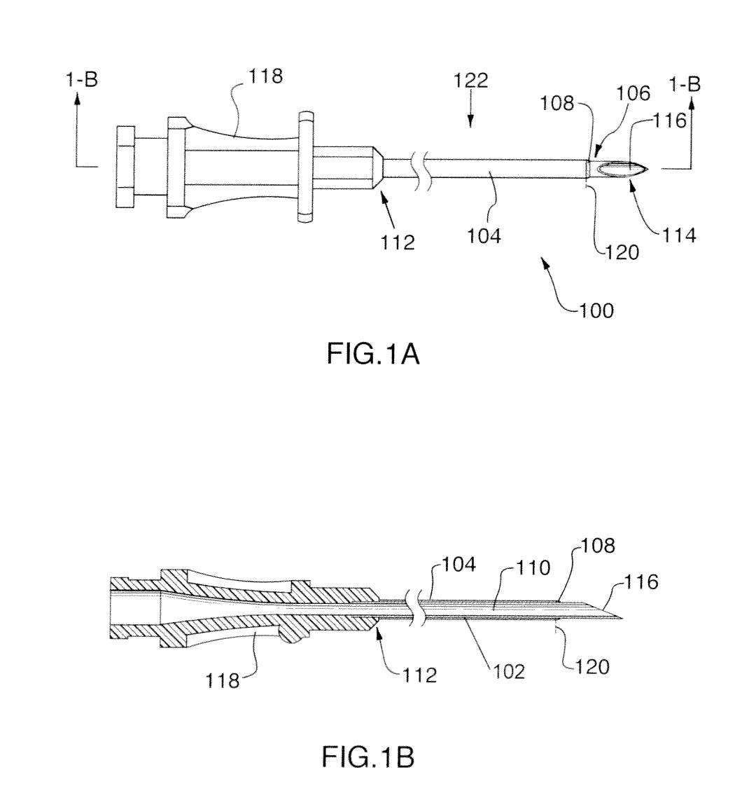

Referring first to FIGS. 1A and 1B, a first embodiment of an electrosurgical cannula 100 of the present invention is shown. FIG. 1A shows a top view of cannula 100 while FIG. 1B illustrates a sectional view of cannula 100 along line 1B-1B in FIG. 1A. Optionally, cannula 100 is manufactured from an electrically conductive and radiopaque material, and a shaft 102 of cannula 100 is at least partially coated with an electrically insulating material 104. Suitable materials for electrically insulating material 104 include, but are not limited to, parylene, PET and PTFE. Shaft 102 has a fixed length in the illustrated example of FIG. 1. A distal tip portion 106 of shaft 102 remains exposed and electrically conductive. Due to the conductive nature of distal tip portion 106, and for the purposes of this invention, distal tip portion 106 may be described as an active electrode or active tip. Distal tip portion 106 is optionally sharp to facilitate penetration into a patient's body. Alternatively, distal tip portion 106 may be blunt, rounded, straight, beveled, rigidly bent, or may take on other forms depending on the particular application. A radiopaque band 108, described in greater detail below, is positioned so as to distinguish active tip 106 from insulated region 122 of shaft 102 under X-ray fluoroscopic imaging.

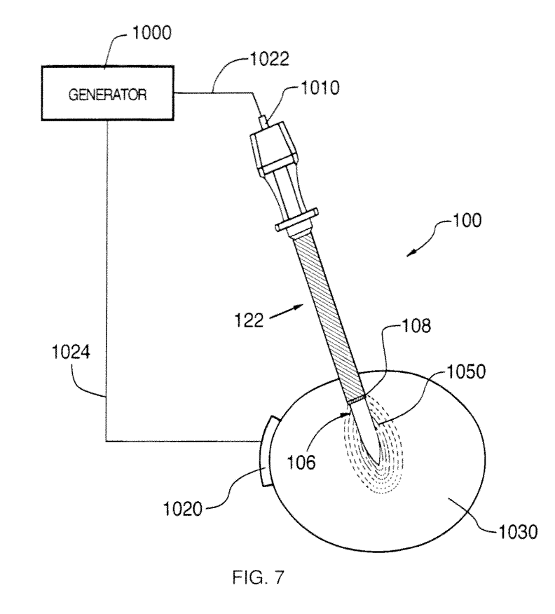

Shaft 102 optionally defines a lumen 110 extending longitudinally from a proximal region 112 to a distal region 114 of cannula 100. In this first embodiment, distal tip portion 106 defines an aperture 116 in communication with lumen 110 of shaft 102. The combination of lumen 110 and aperture 116 allows for the introduction of a fluid or other material into a patient's body. Cannula 100 optionally further comprises a hub 118 located at or adjacent to proximal region 112. Hub 118 is optionally manufactured from ABS (Acrylonitrile Butadiene Styrene) or a similar material and may be attached to shaft 102 using various methods, including but not limited to insert molding, gluing and other forms of bonding. In the context of the present invention, the term hub indicates a fitting or any other means of facilitating a secure connection between separate components such as a cannula and a probe. As such, hub 118 is optionally structured to cooperatively engage and mate with a probe, stylet or other device which may be introduced into shaft 102. In those embodiments that comprise a hub, lumen 110 is optionally sized to simultaneously accommodate a stylet, probe or other device as well as a diagnostic or therapeutic agent. The diagnostic or therapeutic agents may include, but are not limited to, contrast or other chemical agents, pharmaceuticals, and biological agents. In other embodiments, lumen 110 may be designed to receive a probe, stylet or other device without having sufficient space to accommodate a diagnostic or therapeutic agent. In such embodiments, the probe, stylet or other device may be removed from lumen 110, allowing for injection of a diagnostic or therapeutic agent if so desired. It should be noted that, while cannula 100 has been described as having a single lumen, alternate embodiments with no lumen or more than one lumen are also envisioned. Likewise, although this embodiment depicts a cannula with a single aperture, more than one aperture may be disposed along the cannula and the one or more apertures may be disposed at various locations along cannula 100 and are not limited to the locations shown in the appended drawings. Shaft 102 may be generally rigid and substantially entirely straight, as in the example of FIG. 1, for applications requiring insertion along a relatively straight path, or, as illustrated in FIG. 5, shaft 102 may include a curved or rigidly bent distal portion 620 for controlled maneuvering. A doctor or other user can maneuver a generally rigid cannula having a rigidly bent distal portion 620 by, for example, rotating it about the longitudinal axis of shaft 102 during insertion. In contrast to substantially flexible devices such as catheters or wire leads, which may be deflected from a desired insertion path by certain anatomical structures, having a generally rigid shaft allows, for example, for controlled insertion of the cannula along a desired path.

In the first embodiment shown in FIG. 1, radiopaque band 108 is located adjacent a distal edge 120 of electrically insulating material 104, thus allowing a user to distinguish active tip 106 using radiographic imaging techniques. In the illustrated embodiment, band 108 is shown located distal to insulation distal end 120. In alternate embodiments, band 108 is located under insulating material 104 proximally adjacent to insulation distal end 120. In the embodiment shown in FIG. 1, the radiopaque band substantially completely circumscribes shaft 102. In alternate embodiments, band 108 may only partially circumscribe shaft 102. For example, in some embodiments (not shown), the radiopaque band 108 may traverse about 180° of the circumference of shaft 102. In such embodiments, the radiopaque band may be located on the same side of cannula 100 as the opening of aperture 116 or on the opposite side, thus allowing a user to more precisely determine the location of the opening of aperture 116.

Suitable materials for radiopaque band 108 include, but are not limited to, high-density metals such as platinum, iridium, gold, silver, tantalum, palladium or their alloys, or radiopaque polymeric compounds. Such materials are highly visible under fluoroscopic imaging and are therefore visible even at minimal thicknesses. In the embodiment depicted in FIG. 1, radiopaque band 108 has substantially the same outer diameter as electrically insulating material 104, in order to avoid increasing the force required to insert the cannula into a patient's body.

Radiopaque band 108 is optionally laser welded to shaft 102, thus improving the heat resistance of the band-to-cannula bond and allowing the cannula to withstand multiple thermal cycles, as may be experienced when using cannula 100 in conjunction with electrosurgical procedures.

Alternatively, radiopaque band 108 may be applied using a number of techniques known in the art, including but not limited to vapor deposition, ion implantation, ion-bombardment, dip coating, metal plating, welding, soldering and electro plating. In addition, in embodiments wherein radiopaque band 108 is manufactured from a material such as platinum iridium, the band may be fused onto shaft 102. Radiopaque band 108 optionally has a width of about 1 to about 2 mm (approximately 0.04-0.08 inches) and, in some embodiments, has a width of about 1.2 to about 1.3 mm (approximately 0.45-0.05 inches), but this invention may be practiced with radiopaque bands of various widths and is not limited to the specific widths described in conjunction with this first embodiment.

Cannula 100 may be manufactured out of any number of suitable materials, including but not limited to stainless steel, titanium, nitinol or other radiopaque materials in order to impart varying degrees of flexibility, strength, and radiopacity to the device. Cannula 100 is optionally about 18-22 AWG and about 5-10 cm (approximately 2-4 inches) in length and active tip 106 is optionally about 2-10 mm (approximately 0.075-0.4 inches) in length. However, cannula 100, as well as active tip 106, may be designed in a variety of gauges and lengths and the invention is not limited in this regard. In some embodiments, shaft 102 is manufactured from a single radiopaque material resulting in active tip portion 106 and insulated region 122 having substantially the same radiopacity.

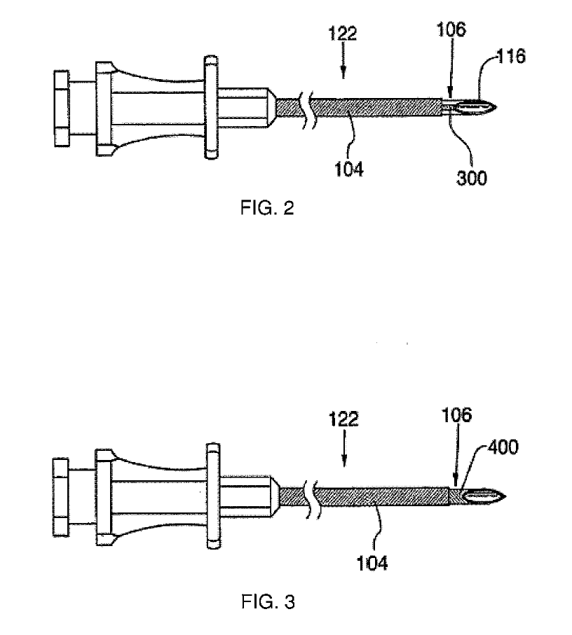

In addition to the band shown in FIGS. 1A and 1B, radiopaque markers of various shapes and patterns may be applied on selected portions of cannula 100 by, for example, the use of various masking techniques as known in the art. Specific patterns of radiopacity can be selected that, while distinguishing distal tip portion 106 from insulated region 122, will also allow one or more of the orientation and position of cannula 100, and the position of aperture 116, to be discerned by inspection of the fluoroscopic image. FIGS. 2-4 illustrate various exemplary embodiments of patterns of radiopacity that may be adopted in accordance with this invention. It will be understood by persons skilled in the art that other shapes and patterns may be adopted as well, and the invention is not intended to be limited to the specific embodiments shown.

FIG. 2 shows an alternate embodiment of the present invention, wherein a radiopaque marker 300 runs substantially parallel to the long axis of the shaft 102 between insulated region 122 and aperture 116 along active tip 116 thereby indicating the precise location of aperture 116 as well as the length of active tip 106. Variations of this embodiment may include various lengths and widths of radiopaque marker 300.

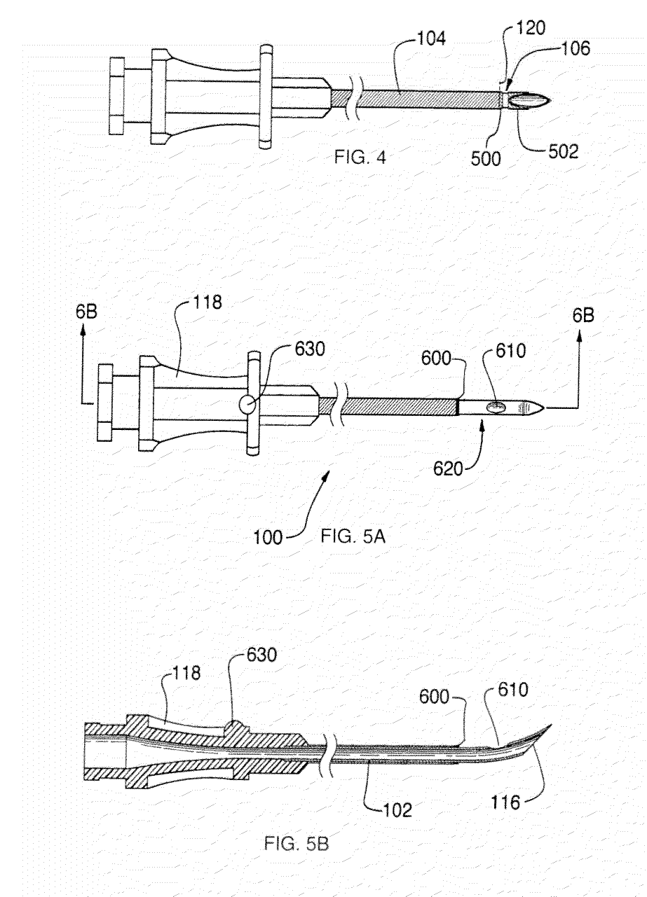

FIG. 3 illustrates yet another embodiment of the present invention wherein a radiopaque marker 400 covers substantially the entire active tip 106 of the cannula. Such an embodiment may be manufactured by, for example, masking insulated region 122 and coating distal tip portion 106 with a suitable radiopaque material, using techniques mentioned above such as vapour deposition and ion bombardment. This embodiment would render the entire active tip 106 readily distinguishable from insulated region 122 under fluoroscopic imaging. In alternate embodiments, a section of distal tip portion 106 may be left uncoated and the invention is not limited in this regard. In an additional embodiment of a radiopaque cannula of the present invention, additionally radiopacity may be imparted to at least a portion of insulated region 122. For example, insulating material 104 may be rendered radiopaque using a number of techniques, including but not limited to vapour deposition, ion-bombardment and ion-implantation. Alternatively, a radiopaque marker may be applied onto a portion of shaft 102 along insulated region 122 prior to the application of insulating material 104. These embodiments would allow insulated region 122 to be distinguishable from active tip 106 under fluoroscopy due to its increased radiopacity.

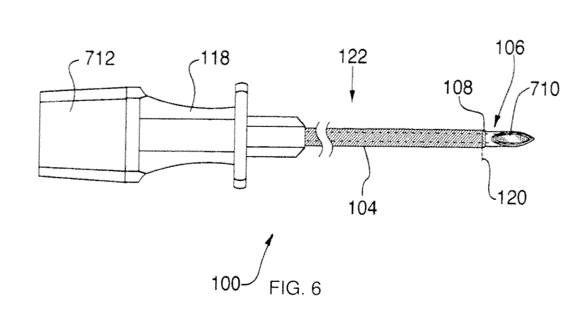

Referring now to the embodiment shown in FIG. 4, the cannula may comprise two radiopaque markers 500 and 502, located adjacent distal edge 120 of insulating material 104 and along a portion of distal tip portion 106, respectively. Such embodiments may be useful to distinguish various regions, for example a distal region and a proximal region, of active tip 106. The specific embodiment shown in FIG. 4 would provide a frame for active tip 106, whereby a user would have precise information regarding the entire length of active tip 106 without having to increase the radiopacity of the entire active tip region. Alternatively, additional radiopaque markers may be located at any desired location of the cannula and the cannula may comprise more than two radiopaque markers.

In the embodiment shown in FIGS. 5A and 5B, cannula 100 comprises a curved or rigidly bent distal tip portion 620 as well as a lateral aperture 610 which may serve as a delivery port for diagnostic or therapeutic agents. With respect to the present invention, the term ‘bent’ is defined to mean having a deviation from a straight line. In addition, the term ‘lateral aperture’ is defined as an aperture or opening defined by a lateral (or radial) surface of the cannula. This may take the form of a rigid bend or a more subtle curve, with various angles of curvature. Lateral aperture 610 induces diagnostic or therapeutic agents introduced into cannula 100 to flow along distal tip portion 620, i.e. parallel and radial to cannula 100, effectively concentrating the diagnostic or therapeutic agents in an area to be treated. Lateral aperture 610 may be disposed on any location of shaft 102, including but not limited to the inner or outer surfaces of the bend of distal tip portion 620, and additional lateral apertures may be present anywhere along the lateral surface of shaft 102. In addition, some embodiments may further comprise an aperture 116, optionally located at an end of distal tip portion 620 (as shown) to maximize delivery of the agent to the target site. Although the embodiment illustrated in FIGS. 5A and 5B shows a cannula with a single lumen, alternate embodiments may comprise a cannula with more than one lumen, whereby each lumen may be connected to one or more apertures. A radiopaque marker 600 may be located on shaft 102 in order to distinguish the active tip 106 and to indicate one or more of the position and orientation of any of the lateral apertures 610 or aperture 116. The specific form and location of radiopaque marker 600 may vary and may, for example, resemble any of the aforementioned embodiments and their equivalents. This particular embodiment of the device of the present invention, comprising a lateral aperture, is especially useful for targeted delivery of anaesthetic to a nerve selected for lesioning. In addition, having a bent tip may allow for easier manoeuvrability of the cannula through a patient's body. Although the embodiment shown in FIGS. 5A and 5B comprises a bent tip, cannulae with straight tips or varying degrees of curvature may also be used in conjunction with one or more lateral apertures. In addition, hub 118 may comprise a marker 630 which may be a visual or tactile indicator to enable more accurate positioning of cannula 100. Marker 630 may be located, for example, on the same side of cannula 100 as lateral aperture 610 or on the opposite side, thus enabling a user to determine the location of lateral aperture 610 after cannula 100 has been inserted into a patient's body.

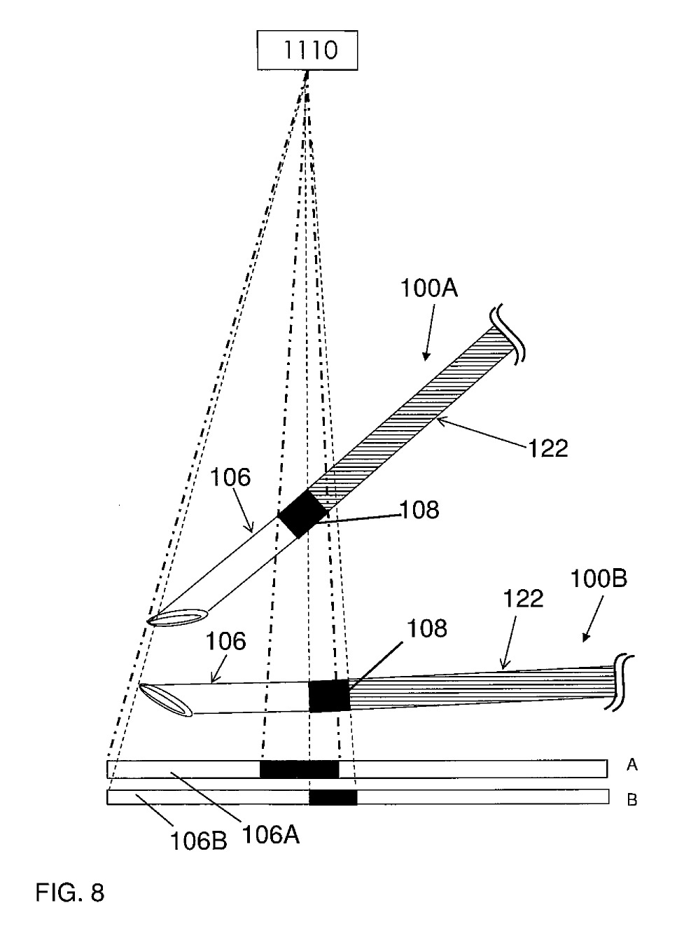

Referring now to FIG. 6, a further embodiment of cannula 100 comprises a lumen into which a removable stylet 710 or probe (not shown) may be inserted. In this embodiment, stylet 710, shown in dotted outline, is adapted to assist in piercing a patient's skin and tissue for entry to a treatment area. As is known in the art, inserting a stylet into a cannula prior to insertion of the cannula into a patient's body helps to ensure that no tissue is forced into a lumen of the cannula by occluding any apertures in the cannula shaft. Stylet 710 optionally comprises a cap 712 adapted to cooperatively engage and mate with hub 118 of cannula 100. Optionally, stylet 710 has a pointed tip, which may be a trocar, conical, bevel, or other shape to allow for easy penetration of tissue when cannula 100 and stylet 710 are introduced into the patient's body. In the embodiment shown, and as described above with respect to FIG. 1, radiopaque band 108 is located adjacent a distal edge 120 of electrically insulating material 104, thereby rendering an insulated region 122 of the shaft distinguishable from active tip 106 under fluoroscopy. However, any of the embodiments described in FIGS. 1-5 may be used in conjunction with stylet 710 and the invention is not limited in this regard.

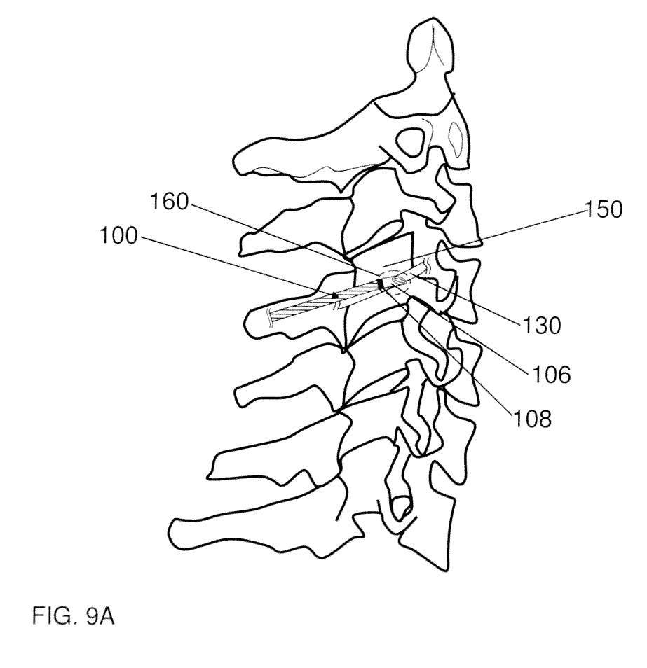

FIG. 7 shows an embodiment of an electrosurgical system incorporating a device of the present invention. The illustrated electrosurgical system comprises an energy generator 1000, a cannula 100 of the present invention, a probe 1010, a reference electrode 1020 and electrical connections 1022 and 1024. In use, and as illustrated in FIG. 7, at least a portion of cannula 100 and probe 1010 are located in a region of a patient's body 1030, while reference electrode 1020 is placed at a location on the surface of body 1030. The components of the electrosurgical system of FIG. 7 will now be described in greater detail.

Energy generator 1000 may be any device capable of operating as a source of energy. In one embodiment, energy generator 1000 is an electrical generator capable of providing high-frequency electrical current. Specifically, energy generator 1000 is optionally operable in a radio-frequency (RF) range and is capable of delivering sufficient power at this frequency so as to effectively treat a patient's pain. Such treatment may take the form of an RF denervation procedure, whereby a lesion is created at a specific neural tissue through heat generated by the application of RF energy, as has been described. Other treatments are possible with such a generator, as is known in the art, and the present invention is not limited to being used in conjunction with any specific procedures.

In the embodiment shown in FIG. 7, cannula 100 comprises a radiopaque marker 108 adapted to allow a user to distinguish an electrically insulated region 122 from an active tip 106 using fluoroscopic imaging, as has been described throughout this specification. Cannula 100 is optionally structured so that an electrical connection between probe 1010 and cannula 100 may be facilitated by the physical inter-relationship of these elements. For example, if cannula 100 is manufactured from a conductive material, physical contact between cannula 100 and probe 1010 may be sufficient to allow for a transfer of electrical energy from the probe to the cannula. In other embodiments, other means of transferring energy from probe 1010 to cannula 100 may be utilized.

Reference electrode 1020 is optionally sufficiently large to prevent localized heating on the surface of body 1030 where reference electrode 1020 is placed. In alternate embodiments, probe 1010 may contain two or more separate electrodes, whereby one electrode (the active electrode) may be electrically connected to cannula 100 and a second electrode may act as a reference electrode, replacing reference electrode 1020. In additional embodiments, reference electrode 1020 may be replaced by a reference electrode located on a second probe inserted into the body proximate to probe 1010.

Electrical connections 1022 and 1024 are any means of conveying or transmitting energy from generator 1000 to probe 1010 and from reference electrode 1020 to generator 1000. For example, electrical connections 1022 and 1024 may comprise electrical cables along with associated connectors for interfacing with generator 1000, probe 1010 and reference electrode 1020. Various other means of electrical coupling are possible and the invention is not limited in this regard.

In general, high frequency electrical current flows from generator 1000 via electrical connection 1022 to probe 1010 and via probe 1010 to active tip 106. This delivery of energy results in electrical stimulation or high frequency heating of tissue in the region surrounding active tip 106. If the tissue surrounding active tip 106 comprises one or more neural structures, the formation of a lesion 1050 may lead to pain relief due to the denervation of said neural structures.

The disclosed cannula is particularly useful for procedures where precise lesioning of the area to be treated is critical. The radiopaque marker adds an element of safety by allowing an operator to reduce unnecessary exposure of energy to tissue that should not be ablated. In facet joint denervation, for example, it is critical that certain nerves, specifically those of the sympathetic chain, are not damaged during the treatment procedure. A radiofrequency treatment procedure, using a device as disclosed herein, may optionally be performed as follows: With a patient lying prone on a radiolucent table, a cannula as presently disclosed, along with a stylet disposed within a lumen of the cannula, is inserted and positioned parallel to the target nerve to be lesioned. Under fluoroscopic guidance, a radiopaque marker located on the cannula may assist in positioning the cannula due to the improved visualization afforded by the radiopaque marker. In particular, the radiopaque marker may assist in positioning the active tip 106 at a specific location relative to one or more anatomical landmarks as described in greater detail herein below. Once positioned, the stylet is removed and replaced by a radiofrequency probe, and the target nerve is located by sensory stimulation. At this point, the position of the active tip of the cannula may be verified using the radiopaque marker as a guide. Finally, following a test for motor stimulation as an added safety measure, energy is delivered from an energy generator through the probe to the active tip of the cannula in order to create a lesion about the target nerve.

In addition to improving treatment safety, it is possible for a radiopaque marker to assist in the visualization of lesion length and to thereby improve treatment efficacy by achieving adequacy of target coverage with minimal repositioning. In some particular applications, radiopaque marker 108 assists in performing sequential lesions using a “tip to tail” repositioning technique, described herein below.

In some embodiments of the treatment procedure, anaesthetic or other diagnostic or therapeutic agents may be injected through the cannula. In such embodiments, a radiopaque marker may be useful in determining the location of any apertures present on the cannula in order to effectively direct the injection of any such agents to the appropriate location. The method aspect of the present invention also provides for the insertion of multiple cannulae of the present invention over the course of a treatment procedure, whereby any or all of the cannulae may be positioned under fluoroscopic guidance, as described above. There are also other possible embodiments of the method using electrosurgical devices that do not require the removal of a stylet and the subsequent insertion of an ablation probe. Such possible embodiments include devices that nave a single insertable element that functions both as a stylet and an electrically powered probe, as well as other variations. In all of the embodiments described herein, the radiopaque marker may be used to improve accuracy of cannula positioning, to assist in the determination of lesion length and to achieve adequacy of target coverage.

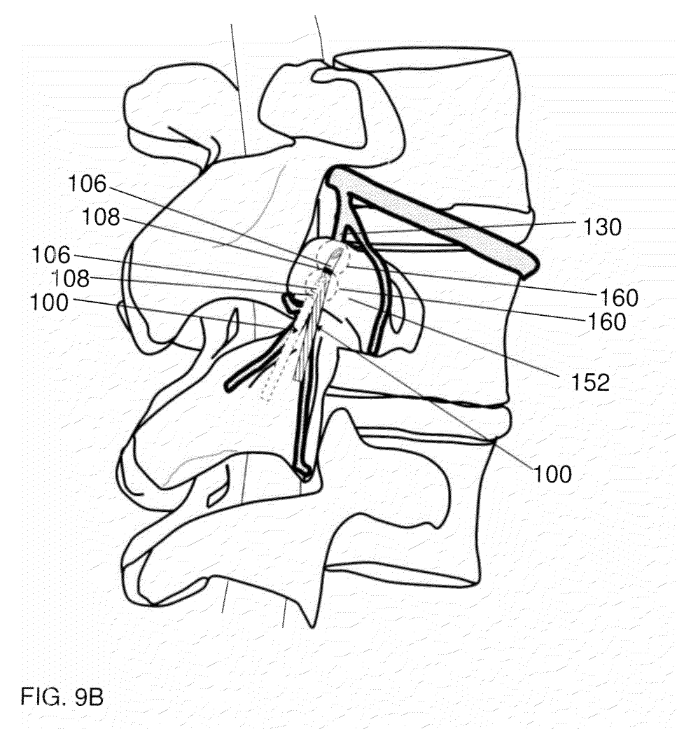

Advantageously, marker 108 can also be used to indicate the orientation of active tip 106. This feature can be used to position the electrically exposed region 106 substantially parallel and adjacent to a targeted nerve branch. For example, as illustrated in FIG. 8, having cannula 100A/B orientated at different angles relative to an imaging source, such as a fluoroscopic x-ray source, will result in an apparent difference in length of active tip 106 when viewed on a two-dimensional display (showing a projection of the two-dimensional projection of the cannula). A physician can use marker 108 to delineate an active tip 106 by being able to see where it ends and to thereby determine the orientation of the active tip. FIG. 8 illustrates X-ray source 1110 projecting images of cannulae 100A and 100B onto corresponding surfaces A and B, intended to represent the linear dimensions of the images as would be seen on a display of a fluoroscopic system. When active tip 106 is substantially perpendicular to the beam projected by X-ray source 1110, as is the case with cannula 100B, the apparent length of active tip 106, as would be shown on the display, is indicated by projection 106B. When active tip 106 is substantially oblique relative to the X-ray beam, as illustrated by cannula 100A, the apparent length of the active tip, indicated by projection 106A, is less than the apparent length when the cannula is perpendicular to the beam. Thus, the apparent length of active tip 106, as shown on the fluoroscopic display, will be shortest when the marker is substantially oblique to the X-ray beam, thereby allowing a user to determine the orientation of the cannula by visualizing the active tip 106 and the radiopaque marker 108.

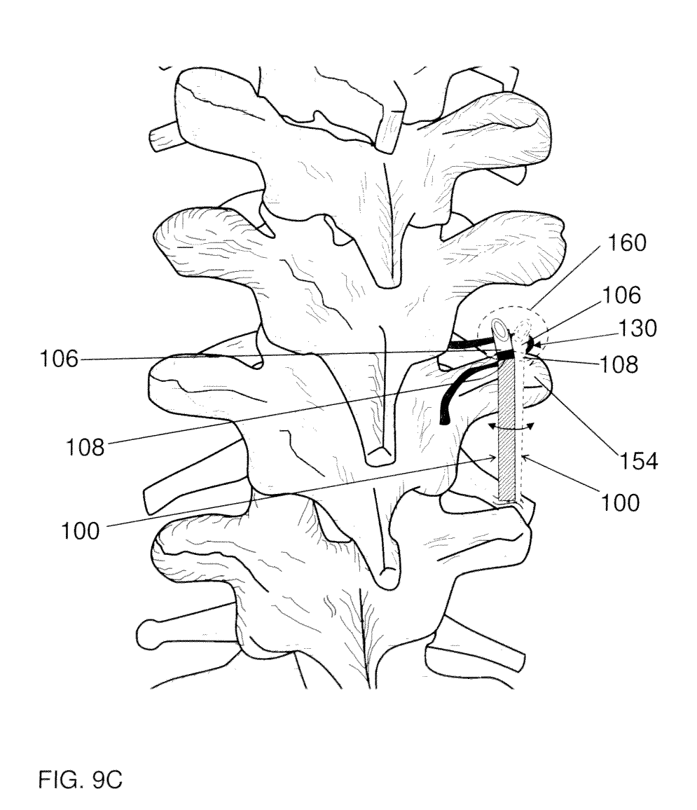

In some embodiments, the method comprises using fluoroscopy or other radiographic imaging techniques to visualize one or more anatomical landmarks in order to facilitate positioning the electrically exposed region 106 proximate to a neural structure having a location definable relative to the fluoroscopically visible anatomical landmarks. Examples of using landmarks in this manner can be seen in FIGS. 9A to 9C which show cannulae positions for lesioning medial branch nerves 130 at various levels of the spine, where the nerves have known approximate locations relative to certain anatomical structures. More specifically, FIGS. 9A, 9B and 9C show cannulae lesioning positions with an active tip 106 positioned relative to the cervical, lumbar and thoracic vertebrae, respectively, of a patient, resulting in electrically exposed regions 106 being adjacent to the target neural structures.

FIG. 9A illustrates an example of a single lesion 160 in the cervical region. Medial branch nerve 130 is located relative to articular pillar 150, which therefore serves as an anatomical landmark. Using an embodiment of a cannula of the disclosure allows a user to determine the length of active tip 106 by visualizing marker 108 and to thereby position active tip 106 relative to articular pillar 150 at a location which would most effectively enable lesioning of the medial branch. In the lumbar region, transverse process 152 (FIG. 9B) may function as such an anatomical landmark while transverse process 154 (FIG. 9C) can be used in the thoracic region.

In the examples of the cervical region (FIG. 9A) and the thoracic region (FIG. 9C), the position of a medial branch nerve 130 relative to a corresponding anatomical feature can vary from vertebrae to vertebrae and from patient to patient but a skilled physician will know the approximate position relative to the anatomical landmark. In the example of the lumbar region (FIG. 9B), a medial branch nerve 130 is normally positioned along a corresponding transverse process 152 in a manner known to a skilled physician. When appropriate, a physician can use nerve stimulation, known to those skilled in the art, in any of the spinal regions to ensure a nerve is targeted correctly.

As illustrated in the example of FIG. 9B, showing the lumbar region of a patient's spine, the radiopaque marker 108 can also be used in tip-to-tail lesioning techniques in which smaller lesions 160 are combined end-to-end to form a larger strip lesion. In one example, when a first lesion is made, the position of the proximal end of active tip 106 is determined by visualizing the radiopaque marker 108. Subsequently, the cannula is retracted proximally to position the distal end (tip) of the active tip 106 at approximately the previous location of the proximal end (tail) of the active tip to provide an extended lesion. Alternatively, after the first lesion is made, the cannula may be advanced or moved distally to position the proximal end of the active tip 106 (whose position is determined by visualizing marker 108) to about where the distal end of the active tip was previously located to create an extended lesion. Depending on whether cannula 100 is advanced or withdrawn between creating lesions, the cannula of FIG. 9B represented by a broken line could be the first or second position. Depending, for example, on the desired overall length of the complete lesion, there may or may not be overlap of the first and second positions of active tip 106. The choice of position of exposed region 106 for the second lesion is up to the discretion of the physician. In the example of FIG. 9B, there is substantially no overlapping of the two positions of active tip 106. Normally a lesion will extend beyond the end of an exposed electrode, such that when the second position of exposed region 106 does not overlap the first, the resulting lesions may still overlap. It is to be understood that when non-overlapping exposed region 106 positions are employed using a method such as the one described herein, that the distance between the positions should ideally be small enough to result in overlapping lesions at the power levels used.

When making lesions in the thoracic region of FIG. 9C, the location of a targeted medial branch nerve 130 is variable but is within a known target volume. Consequently, cannula 100, which, as illustrated in FIG. 9C, has a bent distal tip portion 106, can be rotated after a first lesion is created to a second position to make a second lesion that combines with the first to result in a larger lesion 160 within the target volume. Marker 106 helps to ensure that distal tip portion 106 is correctly positioned for both lesions relative to transverse process 154. In the example of FIG. 9C, the cannula 100 illustrated by a broken line can represent the first or second position, depending on the initial position and the direction of rotation.

Some of the embodiments of the disclosed cannula 100 may comprise other useful features as well as those mentioned above. For example, in some embodiments, hub 118 may comprise some type of visual or tactile marker in order to enable a user to more accurately position cannula 100 before referring to one fluoroscopic image, such as marker 630 in FIG. 5. In addition, cannula 100 may comprise one or more sensors that may be used to monitor physiological parameters such as temperature and pressure. Temperature monitoring sensors may include, but are not limited to, thermocouples, thermistors and thermometers. Pressure monitoring sensors may include, but are not limited to, pressure transducers and fluid-filled lumens in communication with fluid in a patient's body. Some embodiments of the cannula may comprise some means of monitoring electrical impedance, in order to aid in positioning the cannula within a patient's body. In some or all of the embodiments described in this specification, distal tip portion 106 of cannula 100 may be manufactured as a separate piece and may then be attached by some means to shaft 102. Furthermore, additional radiopaque markers may be associated with cannula 100 at any location and the location of the additional radiopaque marker is not limited to the locations described in these embodiments.

It should be noted that the terms radiopaque band, marker, marking etc. as used herein denote any addition or reduction of material that increases or reduces the radiopacity of the device. Furthermore, the terms probe, cannula, stylet etc. are not intended to be limiting and denote any medical and surgical tools that can be used to perform similar functions to those described. In addition, the invention is not limited to be used in the clinical applications disclosed herein, and other medical and surgical procedures wherein a device of the present invention would be useful are included within the scope of the present invention. Furthermore, the cannulae described herein are not intended to be limited to a specific length or gauge, as has been mentioned.

The embodiments of the cannula described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method for treating pain by delivering energy to a patient's body, comprising:
(i) providing an electrically conductive radiopaque cannula comprising an electrically insulated region, an electrically exposed region and a radiopaque marker for distinguishing the insulated region from the exposed region;
(ii) identifying the radiopaque marker, distinguishing the insulated region from the exposed region and positioning the exposed region to locate said exposed region proximate a neural structure; and
(iii) delivering energy through the cannula to said neural structure to treat pain.

2. The method of claim 1, wherein the neural structure is located proximate to or within a facet joint of a patient's spine.

3. The method of claim 1, wherein the energy is delivered directly to said neural structure to ablate said neural structure.

4. The method of claim 1, wherein step (ii) further comprises positioning said exposed region substantially parallel and adjacent to said neural structure.

5. The method of claim 1, wherein step (ii) further comprises visualizing one or more anatomical landmarks associated with a location of said neural structure to facilitate the positioning of said exposed region to locate said exposed region proximate to said neural structure.

6. The method of claim 1, wherein in step (iii) the energy is delivered to create a first lesion, and wherein said method further comprises:
(iv) determining the locations of a proximal end and of a distal end of the electrically exposed region;
(v) repositioning the cannula such that said distal end of said electrically exposed region is positioned substantially at the location in which said proximal end of said electrically exposed region had been determined to be located in step (iv); and
(vi) delivering energy through the cannula to said neural structure to create a second lesion.

7. The method of claim 1, wherein in step (iii) the energy is delivered to create a first lesion, and wherein said method further comprises the steps of:
(iv) determining the locations of a proximal end and of a distal end of the electrically exposed region;
(v) repositioning the cannula such that said proximal end of said electrically exposed region is positioned substantially at the location in which said distal end of said electrically exposed region had been determined to be located in step (iv); and
(vi) delivering energy through the cannula to said neural structure to create a second lesion.

8. The method of claim 1 wherein a position of said electrically insulated region, relative to said electrically exposed region, is fixed.

9. The method of claim 1 for allowing a user to determine the orientation of the cannula by visualizing the exposed region and the radiopaque marker.

10. A radiopaque cannula for insertion into a patient's body, the cannula comprising:
a radiopaque and electrically conductive elongate member having a proximal end and a distal end and defining a lumen therebetween, the elongate member including an electrically exposed region and an electrically insulated region; and
a radiopaque marker associated with the elongate member, the radiopaque marker being visible under radiographic imaging and being located for distinguishing the exposed region from the insulated region.

11. The cannula of claim 10, wherein said radiopaque marker is located substantially adjacent a distal end of the insulated region.

12. The cannula of claim 10, wherein said radiopaque marker comprises a material selected from the group consisting of platinum, iridium, gold, silver, tantalum, palladium and alloys thereof.

13. The cannula of claim 10, wherein at least a portion of said distal end of said elongate member is bent.

14. The cannula of claim 10, wherein said distal end of said elongate member defines an aperture in communication with said lumen.

15. The cannula of claim 10, further comprising a lateral aperture in communication with said lumen.

16. The cannula of claim 10, wherein said cannula is operable to connect to an energy source.

17. The cannula of claim 10, wherein the electrically insulated region comprises a coating of an electrically insulating material disposed external to the elongate member along at least a portion of the elongate member.

18. The cannula of claim 17, wherein said electrically insulating material comprises a material selected from the group consisting of parylene, PET and PTFE.

19. The cannula of claim 10, wherein the marker is affixed to the elongate member by a laser weld.

20. The cannula of claim 10, wherein the radiopaque marker comprises a band that substantially completely circumscribes the elongate member.

21. The cannula of claim 10, wherein said radiopaque marker comprises a material of a greater radiopacity than the material comprising said elongate member.

22. The cannula of claim 10, wherein said elongate member is substantially rigid.

23. A cannula for insertion into a patient's body, said cannula comprising:
an electrically conductive radiopaque elongate member comprising an electrically insulated region and an electrically exposed region; and
a means for improving radiographic visualization of at least a portion of said elongate member;
said means for improving radiographic visualization being located for distinguishing the exposed region from the insulated region.
